(19) United States
(12) Patent Application Publication
(10) Pub. No.: US 2026/0102499 A1
(43) Pub. Date: Apr. 16, 2026

(54) COMPOSITIONS WITH REDUCED NITROSAMINE IMPURITIES

(71) Applicant: INVENTIA HEALTHCARE LIMITED, Mumbai, Maharashtra (IN)

(72) Inventors: Vaibhavi SHAH, Mumbai, Maharashtra (IN); Nirav KAMDAR, Mumbai, Maharashtra (IN); Bhooma RAGHAVAN, Mumbai, Maharashtra (IN)

(21) Appl. No.: 19/114,030

(22) PCT Filed: Sep. 26, 2023
(86) PCT No.: PCT/IN2023/050888

§ 371 Date: Mar. 21, 2025

(30) Foreign Application Priority Data
Sep. 27, 2022 (IN) 202221055262

(51) Int. Cl. A61K 47/18; A61K 9/20; A61K 9/28; A61K 45/06

(52) U.S. Cl. CPC A61K 47/18; A61K 9/2095; A61K 9/282; A61K 9/2866; A61K 9/2893; A61K 45/06

(57) ABSTRACT

The present invention relates to storage-stable pharmaceutical compositions comprising a drug substance containing amine, and at least one excipient, wherein the nitrosamine impurities in the said compositions are within acceptable levels. The nitrite content of the storage-stable pharmaceutical compositions is not more than about 2 ppm. Further, the present invention also provides a process for the manufacture of the said compositions.

### FIELD OF THE INVENTION

The present invention relates to storage-stable pharmaceutical compositions comprising a drug substance containing amine, and at least one excipient, wherein the nitrosamine impurities in the said compositions are within acceptable levels. Further, the present invention also provides a process for the manufacture of the said compositions.

### BACKGROUND OF THE INVENTION

Nitrosamine impurities are potential genotoxic agents, and are classified as possible human carcinogens by the International Agency for Research on Cancer (IARC). It is essential to control mutagenic and carcinogenic nitrosamine impurities in pharmaceutical compositions, at or below a level such that there would be a negligible human cancer risk associated with the intake and exposure to such impurities.

International regulators such as United States Food and Drug Administration (USFDA), European Medicine Agency (EMA), and Health Canada have published recommendations with respect to ‘acceptable intake’ of nitrosamine impurities. ‘Acceptable intake’ (per day) of nitrosamine impurities varies with the impurity structure, molecular weight, and its toxicity potential. Table 1 illustrates the per day ‘acceptable intake’ of some nitrosamine impurities, as published by Health Canada.

TABLE 1
Acceptable Intake (AI) Levels or Nitrosamine Impurities

| | |
|---|---|
| Nitrosamine Impurity | AI Levels (ng/day)* |

A. Nitrosamine drug substance related impurities (NDSRIs) (nitrosamine
impurities sharing structural similarity to the drug substance)

| | |
|---|---|
| N-nitroso-methylphenidate | 1300.0 |
| N-nitroso-piperidine | 1300.0 |
| N-nitrosomorpholine (NMOR) | 127.0 |
| N-nitroso-duloxetine (NDLX) | 100.0 |
| N-nitroso-4-(methylamino)butyric acid (NMBA) | 96.0 |
| 1-methyl-4-nitrosopiperazine (MNP) | 96.0 |
| N-nitroso-varenicline (NNV) | 37.0 |
| 7-Nitroso-3-(trifluoromethyl)-5,6,7,8- | 37.0 |
| tetrahydro[1,2,4]triazolo-[4,3-a]pyrazine (NTTP) | |
| N-nitroso-1,2,3,6-tetrahydropyridine (NTHP) | 37.0 |
| N-nitroso-rasagiline | 18.0 |
| N-nitroso-nortriptyline (NNORT) | 8.0 |

B. Other Nitrosamine Impurities

| | |
|---|---|
| N-nitroso-dimethylamine (NDMA) | 96.0 |
| N-nitroso-diethylamine (NDEA) | 26.5 |
| N-nitroso-diisopropylamine (NDIPA) | 26.5 |
| N-nitroso-ethylisopropylamine (NEIPA) | 26.5 |
| N-nitroso-dibutylamine (NDBA) | 26.5 |
| N-nitroso-dipropylamine (NDPA) | 26.5 |
| N-methyl-N-nitroso-phenethylamine (NMPEA) | 8.0 |

‘USDFA in its Guidance Document titled ‘Control of Nitrosamine Impurities in Human Drugs’ states that nitrosamine impurities are generated when secondary, tertiary, or quaternary amine groups react with nitrous acid (nitrosating agent). Nitrosamine impurities in compositions are generated at the time of their manufacture and/or storage, or may be carried forward from their components (drug substance, excipients, solvents, and packaging materials).

Secondary, tertiary, or quaternary amine groups may be present in the components of the composition (e.g drug substance containing amine), or compounds containing such amine groups may be carried forward into the components and/or their compositions from starting materials (intermediates, reagents, catalysts) and degradants.

Nitrous acid, being unstable, is formed in situ from nitrites (NO₂) and nitrates (NO₃) (precursor to nitrites), under acidic conditions. Nitrites and nitrates, being commonly used reagents, are often carried forward into the components and/or their compositions.

‘Acceptable intake’ of a nitrosamine impurity, when applied to the maximum daily dose of a drug, provides ‘acceptable levels’ of the said nitrosamine impurity in pharmaceutical compositions containing the said drug. ‘Acceptable levels’ of a nitrosamine impurity are expressed in parts per million (ppm) or parts per billion (ppb), of the drug (by weight). For example, for varenicline (which contains amine), the ‘acceptable intake’ of the N-nitroso-varenicline (NNV) (nitrosamine impurity of the NDSRI type) is 37 ng/day, and the ‘acceptable level’ of NNV is 18.5 ppm based on a maximum daily dose of 2 mg for varenicline.

In addition to complying with acceptable levels of nitrosamine, the compositions must also meet the requirements of assay of drug substance containing amine, average cumulative % dissolution of drug substance containing amine, and content of total impurities.

Prior art uses different strategies to reduce formation of nitrosamine impurities in pharmaceutical compositions.

PCT Application No. PCT/IB2021/057245 relates to a process for reduction of N-nitroso dimethylamine impurity in metformin compositions, by using scavengers such as silica, oxygen absorbing materials, and activated carbon, in the storage containers of the said compositions.

PCT Application No. PCT/EP2021/078691 provides a pharmaceutical composition comprising metformin, and an anti-oxidant, free-radical scavenger or reducing agent.

Chinese Application No. CN111686087 relates to tablets of ranitidine, prepared by powder mixing, direct compression, and gastric-soluble film coating. The process avoids excessive heat generation during the production process to reduce the risk of NDMA (nitrosamine impurity) generation.

Indian Application No. 202021039399 and US Patent Application 2022/0079894 relates to reducing the amount of NDMA formed in tablets containing angiotensin II receptor antagonist (ARBs) drugs, gastroesophageal reflux disease (GERD) drugs, or antidiabetic drugs (like metformin) by incorporating desiccant as a moisture absorption agent in the sealed container in which the tablet is stored.

PCT Application No. PCT/CN2021/122654 relates to inhibiting or reducing the generation of nitrosamine impurities, and improving the stability of a secondary amine compound (varenicline) in a pharmaceutical composition thereof, by adding a pharmaceutically acceptable acid (pH regulator/acid regulator) to the composition.

It has surprisingly been found that it is possible to provide storage-stable pharmaceutical compositions comprising a drug substance containing amine, and at least one excipient, wherein the nitrosamine impurities in the said compositions are within acceptable levels, when the compositions have nitrite content of less than or equal to 2 ppm.

Further, it has surprisingly been found that the said storage-stable pharmaceutical compositions are prepared by processes, wherein the solvent system used in any of the process steps contains not more than about 20% by weight of water, preferably not more than about 15% by weight of water.

### OBJECTS OF THE INVENTION

The main object of the present invention is to provide storage-stable pharmaceutical compositions comprising a drug substance containing amine, and at least one excipient, wherein the nitrosamine impurities in the said compositions are within acceptable levels.

Yet another object of the present invention is to provide storage-stable pharmaceutical compositions comprising a drug substance containing amine, and at least one excipient, wherein nitrite content in the composition is less than or equal to about 2 ppm.

Yet another object of the present invention is to provide a process for the manufacture of the said pharmaceutical compositions.

### SUMMARY OF THE INVENTION

The present invention relates to storage-stable pharmaceutical compositions comprising a drug substance containing amine, and at least one excipient, wherein the nitrosamine impurities in the said compositions are within acceptable levels. The present invention further provides a process for the manufacture of the said compositions.

### DETAILED DESCRIPTION OF THE INVENTION

Pharmaceutical compositions of the present invention comprise a drug substance containing amine, and at least one excipient, wherein the nitrosamine impurities in the said compositions are within acceptable levels.

The term ‘drug substance containing amine’ as used herein refers to active ingredients containing secondary, tertiary, or quaternary amine groups, or their salts, hydrates, esters, derivatives or solvates thereof.

The term ‘nitrosamine impurity’ or ‘nitrosamine impurities’, as used herein refers to a class of compound(s) having the chemical structure of a nitroso group bonded to an amine R₁—N(—R₂)—N═O, which can be formed by a nitrosating reaction between an amine group and nitrous acid.

The term ‘nitrosamine impurity’ or ‘nitrosamine drug-substance-related impurities (NDSRIs)’ as used herein refers to a nitrosamine impurity or a class of nitrosamine impurities, whose structure is similar to the drug substance containing amine.

The term ‘storage-stable’ or ‘storage-stable compositions’, as used herein, refers to compositions which contain acceptable levels of nitrosamine impurities, when stored at 40° C.±2° C. and 75%±5% relative humidity (40° C./75% RH) for at least 3 to 6 months, or when stored at 25° C.±2° C. and 60%±5% relative humidity (25° C./60% RH) for at least 12 months, or when stored at 30° C.±2° C. and 75%±5% relative humidity (30° C./75% RH) for at least 12 months.

The term ‘solvent system’, as used herein, refers to a single non-aqueous solvent, or a mixture of two or more non-aqueous solvents, or a mixture of at least one non-aqueous solvent and water.

The pharmaceutical compositions of the present invention, comprising a drug substance containing amine, and at least one excipient, are storage-stable, with nitrosamine impurities within acceptable levels.

In an aspect, the said compositions that contain reduced levels of nitrite and/or nitrate, are storage-stable with nitrosamine impurities within acceptable levels.

The said compositions are manufactured by processes, wherein the solvent system used in any of the process steps contains not more than about 20% by weight of water.

In an aspect, the solvent system used in the process step(s) contains about 0% to about 20% by weight of water.

In another aspect, the storage-stable pharmaceutical compositions comprise drug substance containing amine, at least one excipient, and nitrosamine impurity within the acceptable level, and are manufactured by a process that uses a solvent system containing about 0% to about 20% by weight of water.

In another aspect, the compositions of the present invention comprise excipient(s) containing reduced levels of nitrite and/or nitrate.

In an embodiment, the nitrite content of the said compositions is not more than about 2 ppm.

In another embodiment the nitrite content of the said compositions is less than or equal to about 2 ppm, or from about 0 ppm to about 2 ppm, or from 0.01 ppm to about 2 ppm, or from about 0.05 ppm to about 1.75 ppm, or from about 0.1 ppm to about 1.5 ppm, or from about 0.25 ppm to about 1.25 ppm.

In yet another embodiment the nitrate content of the said compositions is not more than about 5 ppm.

In yet another embodiment, the nitrate content of the said compositions is less than or equal to about 5 ppm, or from about 0 ppm to about 5 ppm, or from about 0.1 ppm to about 5 ppm, or from about 0.5 ppm to about 4.5 ppm, or from about 1 ppm to about 4 ppm, or from about 1.5 ppm to about 3.5 ppm.

In an aspect, the storage-stable pharmaceutical compositions comprise drug substance containing amine and at least one excipient, wherein the nitrite content of the compositions is not more than about 2 ppm, and the nitrosamine impurity content is within acceptable level.

The process for the manufacture of the storage-stable compositions of the present invention comprises steps of mixing, compression, and coating, wherein the coating process step uses a solvent system containing 0% to 20% by weight of water

The process for the manufacture of the storage-stable compositions of the present invention comprises steps of:
a) mixing a drug substance containing amine and at least one excipient, to provide a mixture,
b) optionally granulating the mixture from step a) followed by lubrication,
c) compressing the mixture from step a) or granules from step b) into tablets, or filling the mixture from step a) or granules from step b) into capsules
d) coating the tablets or capsules, from step c), using a solvent system;
wherein the said solvent system contains about 0% to about 20% by weight of water.

In an embodiment, the process for the manufacture of storage-stable compositions, comprises steps of:
a) mixing a drug substance containing amine and at least one excipient, to provide a mixture,
b) granulating the mixture from step a) using a solvent system,
c) lubricating the granules from step b),
d) compressing the granules from step c) into tablets, or filling the granules from step c) into capsules,
wherein the said solvent system contains about 0% to about 20% by weight of water.

In an embodiment, the process for the manufacture of storage-stable compositions, comprises steps of:
a) mixing a drug substance containing amine and at least one excipient, to provide a mixture,
b) granulating the mixture from step a) using a solvent system,
c) lubricating the granules from step b),
d) compressing the granules from step c) into tablets, or filling the granules from step c) into capsules,
e) optionally coating the tablets or capsules, from step d), using a solvent system;
wherein the said solvent system contains about 0% to about 20% by weight of water.

In another embodiment, the said storage-stable compositions are prepared by processes, wherein the solvent system used in any of the process steps contains about 0% to about 20% by weight of water, preferably about 1% to about 15% by weight of water.

‘Granulation’ may be carried out by wet granulation, fluidized bed granulation, rapid mixer granulation, high-shear mixer granulation, centrifugal wet granulation, steam granulation, spray drying granulation, melt granulation, melt extrusion, spheronization, freeze-granulation, thermal adhesion granulation, foam granulation, solvent evaporation, co-crystallization, co-precipitation, dry granulation, moisture-activated dry granulation, pneumatic dry granulation, roll compaction, slug compaction, co-milling, or a combination thereof.

‘Coating’ may be film-coating, seal-coating, barrier-coating, drug-coating, modified release coating, taste-masking coating, color coating, aesthetic coating, moisture-barrier coating, light-barrier coating, stabilizer-coating, or a combination thereof.

In an embodiment, the process for the manufacture of the said compositions is devoid of any process step that uses a solvent system.

In another embodiment, the process for the manufacture of the said compositions contains a process step that uses a solvent system.

Solvent systems, used in manufacture of the storage-stable compositions, are selected from one or more non-aqueous solvents, or a mixture of one or more non-aqueous solvents and water. Non-aqueous solvents may be alcoholic solvents such as methanol, ethanol, and isopropanol (IPA), esters solvents such as ethyl acetate, ketone solvents such as acetone, chlorinated hydrocarbon solvents such as dichloromethane and trichloroethane, or mixtures thereof.

In an aspect, the solvent system, used in the manufacture of the said compositions, contains about 100% by weight of one or more non-aqueous solvents.

In an embodiment, the solvent system, used in the manufacture of the said compositions, contains a mixture of isopropanol and dichloromethane, or a mixture of acetone and ethyl acetate.

In another aspect, the solvent system, used in the manufacture of the said compositions, contains a mixture of one or more non-aqueous solvents and water, wherein water is not more than about 20% by weight of the solvent system, preferably not more than about 15% by weight of the solvent system.

In another embodiment, the solvent system, used in the manufacture of the said compositions, is a mixture of isopropanol and water, or a mixture of ethanol and water, or a mixture of methanol and water, or a mixture of acetone and water, wherein water is not more than about 20% by weight of the solvent system.

In yet another embodiment, the solvent system, used in the manufacture of the said compositions, is a mixture of at least one non-aqueous solvent and water, in a weight ratio ranging from 4:1 to 1:0.

In yet another embodiment, the solvent system, used in the manufacture of the said compositions, is a mixture of at least two non-aqueous solvents, in a weight ratio ranging from 20:1 to 1:20

In a preferred embodiment, the solvent system used in the manufacture of the said storage-stable compositions has a nitrite content of less than or equal to about 2 ppm and/or has a nitrate content of less than or equal to about 5 ppm.

Excipient(s), in the storage-stable pharmaceutical compositions of the present invention, may be selected from diluents, disintegrants, binders, surfactants, glidants, lubricants, coating agents, plasticizers, and colorants.

Diluent(s) may be selected from microcrystalline cellulose, silicified microcrystalline cellulose, powdered cellulose, carboxymethylcellulose calcium, microfine cellulose, maltodextrin, starch, calcium phosphate, dibasic calcium phosphate, tribasic calcium phosphate, mannitol, sorbitol, lactose, and mixtures thereof.

Diluent(s) may be present in concentrations ranging from about 5% to about 99%, preferably from about 20% to about 95%, more preferably from about 40% to about 95%, and most preferably from about 60% to about 95% by weight of the compositions.

In an embodiment, diluent(s) may be present in concentrations ranging from about 70% to about 99%, preferably from about 75% to about 95%, and more preferably from about 80% to about 95% by weight of the compositions.

In an embodiment, the storage-stable compositions of the present invention are free of maltodextrin.

Disintegrants may be selected from selected from sodium starch glycolate, starch, pregelatinized starch, crospovidone, hydroxy propyl cellulose (low substituted), and croscarmellose sodium.

Disintegrant(s) may be present in concentrations ranging from about 1% to about 20% by weight of the composition.

Binders may be selected from hydroxypropyl methyl cellulose (HPMC), hydroxypropyl cellulose (HPC), methylcellulose, hydroxyethyl 30 cellulose, starch, polyvinylpyrrolidone (PVP), polyvinyl alcohol, waxes, fatty alcohols, polyethylene glycol, carbomer, gums, alginates, polymethacrylates, and mixtures thereof. Binders can be used in their dry form, or can be dissolved in a suitable solvent system to give a granulating liquid which is used in the manufacture of granules.

Binders may be present in concentrations from about 0.1% to about 40%, preferably from about 0.1% to about 30%, and more preferably from about 0.1% to about 20% by weight of the pharmaceutical composition.

In an aspect, the storage-stable compositions are free of binder(s).

In another aspect, the storage-stable compositions are free of surfactant(s).

Lubricant(s) may be present in concentrations from about 0.25% to about 5% by weight of the composition, and are selected from those known in the art such as magnesium stearate, calcium stearate, stearic acid, sodium stearyl fumarate, sodium benzoate, palmitic acid, talc, and glyceryl behenate.

Glidant(s) may be present in concentrations from about 0.1% to about 10% by weight of the composition, and are selected from those known in the art such as colloidal silicon dioxide, hydrated silicon dioxide, light anhydrous silicic acid, aluminum silicate, titanium oxide, stearic acid, and talc.

Coating agent(s) are selected from those known in the art such as hydroxypropyl methylcellulose, hydroxypropyl cellulose, ethyl cellulose, cellulose acetate, cellulose acetate phthalate, hydroxypropyl methyl cellulose phthalate, hydroxypropyl methyl cellulose acetate succinate, polyacrylic acid, polymethacrylates, polyvinyl alcohol, polyvinyl acetate, and polyvinyl acetate phthalate.

Plasticizers are selected from those known in the art such as triacetin, triethyl citrate, tributyl citrate, diethyl phthalate, dibutyl sebacate, diethyl phthalate, polyethylene glycol, propylene glycol, glycerol monocaprylocaprate, and acetylated mono- and di-glycerides.

Alternatively, the coating may be applied by using commercially available film coating pre-mixtures such as Opadry™ which contains the coating agent and optionally plasticizer(s), glidant(s), lubricants(s), surfactant(s), and/or colorant(s).

In an aspect, the storage-stable compositions of the present invention are free of stabilizers such as antioxidants, pH modifiers, amino acids, and sugar alcohols.

In an embodiment, the storage-stable compositions of the present invention are free of antioxidants such as ascorbic acid, sodium ascorbate, ascorbyl palmitate, magnesium ascorbyl phosphate, sodium ascorbyl phosphate, propyl gallate, tocopherol, alpha-tocopherol, butylated hydroxytoluene, butylated hydroxyanisole, edetic acid, edetate salts, sodium sulfite, sodium thiosulfate, sodium bisulfite, sodium metabisulfite, sodium pyrosulfite, gentisic acid, thioglycerol, thiourea, or mixtures thereof.

In another embodiment, the storage-stable compositions of the present invention are free of organic acidic agents, inorganic acidic agents, organic basic agents, inorganic basic agents and pH modifiers, such as ascorbic acid, fumaric acid, citric acid, malic acid, succinic acid, adipic acid, maleic acid, lactic acid, hydrochloric acid, nitric acid, phosphoric acid, sulfuric acid, tartaric acid, succinic acid, benzenesulfonic acid, oxalic acid, meglumine, sodium carbonate, sodium bicarbonate, potassium bicarbonate, lithium carbonate, potassium carbonate, calcium carbonate, magnesium carbonate, magnesium oxide, sodium citrate, sodium hydroxide, potassium hydroxide, ammonium salts, magnesium aluminum silicate, magnesium aluminometasilicate, magnesium silicate, and mixtures thereof

In yet another embodiment, the storage-stable compositions of the present invention are free of amino acids such as acetyl cysteine, cysteine, glycine, lysine, histidine, leucine, arginine, alanine, asparagine, glutamine, isoleucine, methionine, phenylalanine, proline, serine, threonine, tryptophan, tyrosine, valine, or mixtures thereof.

In yet another embodiment, the storage-stable compositions of the present invention are free of sugar alcohols such as alditol, polyol, mannitol, meglumine, xylitol, isomalt, sorbitol, lactitol, pentitol, arabitol, ribitol, galactitol, erythritol, glycerol, or mixtures thereof.

The said storage-stable compositions comprise a drug substance containing amine and at least one excipient, wherein the excipient has a nitrite content of not more than about 2 ppm and/or a nitrate content of not more than about 5 ppm.

In a preferred embodiment, the nitrite content of the said excipient(s) and the solvent system, used in the said storage-stable compositions, is less than or equal to about 2 ppm, or from about 0 ppm to about 2 ppm, or from about 0.01 ppm to about 2 ppm, or from about 0.01 ppm to about 1.75 ppm, or from about 0.01 ppm to about 1.5 ppm, or from about 0.01 ppm to about 1.25 ppm, or from about 0.01 ppm to about 1 ppm, or from about 0.01 ppm to about 0.75 ppm, or from about 0.01 ppm to about 0.6 ppm, and or from about 0.01 ppm to about 0.5 ppm.

In a preferred embodiment, the nitrate content of the said excipient(s) and the solvent system, used in the said storage-stable compositions, is less than or equal to about 5 ppm, or from 0 ppm to 5 ppm, or from about 0.1 ppm to about 5 ppm, or from about 0.1 ppm to about 4.5 ppm, or from about 0.1 to about 4 ppm, or from about 0.1 to about 3.5 ppm, or from about 0.1 ppm to about 3 ppm, or from about 0.1 ppm to about 2.5 ppm, and or from about 0.1 ppm to about 2 ppm.

In an aspect the storage-stable compositions comprise diluent(s), disintegrant(s) and/or coating agent(s) as the excipient, wherein the excipient has a nitrite content of not more than about 2 ppm and/or a nitrate content of not more than about 5 ppm.

Storage-stable compositions of the present invention can be orally administered in the form of granules, pellets, tablets, capsules, or a combination thereof.

‘Drug substance containing amine’, used in the storage-stable compositions of the present invention, include but are not limited to varenicline, metformin, sitagliptin, duloxetine, hydrochlorothiazide, Beta blockers such as metoprolol and propranolol, ARBs such as valsartan, olmesartan, candesartan, losartan, irbesartan and telmisartan, H2-receptor antagonists such as ranitidine, cimetidine and nizatidine, ticagrelor, rifampin, rifapentine, pioglitazone, and pharmaceutically acceptable salts, hydrates, esters, derivatives or solvates thereof.

‘Drug substance containing amine’ can be in crystalline and/or amorphous form.

In a preferred embodiment, the drug substance containing amine is varenicline, or its salt such as varenicline tartrate, varenicline hydrochloride, varenicline sulphate, varenicline mesylate, or varenicline fumarate.

In storage-stable compositions comprising varenicline or its salts as the drug substance containing amine, the content of nitrosamine impurity-N-nitroso-varenicline (NNV) is not more than about 18.5 ppm, preferably not more than about 18 ppm, and more preferably not more than about 17.5 ppm, by weight of varenicline.

In an aspect, the storage-stable compositions of the present invention comprise varenicline or its salts, and at least one excipient, wherein the nitrosamine (NNV) impurity content is not more than about 18.5 ppm.

In another aspect, the storage-stable pharmaceutical compositions comprise varenicline or its salt, at least one excipient, and not more than 18.5 ppm of N-nitroso-varenicline impurity, wherein the said compositions are manufactured by a process that uses a solvent system containing about not more than 20% by weight of water.

The said varenicline compositions are prepared by processes, wherein the solvent system used in any of the process steps contains about 0% to about 20% by weight of water, preferably from about 1% to about 15% by weight of water.

In an embodiment, the nitrite content of the said varenicline compositions is less than or equal to about 2 ppm and/or the nitrate content of the said compositions is less than or equal to about 5 ppm.

In an embodiment, the nitrite content of the said varenicline compositions is not more than about 2 ppm and/or the nitrate content of the said compositions is not more than about 5 ppm.

In another embodiment, the said varenicline compositions comprise diluents, disintegrants, and/or coating agents as excipients, wherein the said excipients have a nitrite content of less than or equal to about 2 ppm and/or a nitrate content of less than or equal to about 5 ppm.

In another embodiment, the said varenicline compositions comprise diluents, disintegrants, and/or coating agents as excipients, wherein the said excipients have a nitrite content of not more than about 2 ppm and/or a nitrate content of not more than about 5 ppm.

Storage-stable compositions of the present invention may be packed in suitable packs such as blisters, strips, or bottle packs, which use packing materials such as polyvinyl chloride (PVC), polyvinylidene chloride (PVDC), polychlorotrifluroethylene (PCTFE), polypropylene (PP), polyethylene (PE), aluminum, cellulose polymers, or mixtures thereof.

The preferred packs used for the storage-stable compositions are nitrocellulose free.

In an embodiment, the packs may be used with desiccants such as silica gel, activated carbon, clay, and molecular sieves, and/or oxygen absorbers.

Storage-stable compositions of the present invention were evaluated with respect to one or more of the following parameters: Content of drug substance containing amine (assay), nitrite content, nitrate content, content of nitrosamine impurities, content of total impurities, and average cumulative % dissolution of drug substance containing amine.

Content of drug substance containing amine (assay), in the storage-stable compositions, as determined by liquid chromatography, ranges from about 90% to about 110%, preferably from about 92.5% to about 107.5%, and most preferably 95% to about 105% of the label claim.

Nitrite Content and/or Nitrate Content, in the excipient, the solvent system, or storage-stable compositions, may be determined by methods such as liquid chromatography, anion exchange chromatography, ion-pair chromatography, polarographic methods, and capillary electrophoresis. Nitrite and Nitrate levels are expressed in parts per million (ppm) based on the weight of the composition.

Content of Nitrosamine impurity, in the storage-stable compositions, is determined by liquid chromatography. Nitrosamine impurity is expressed in parts per million (ppm) based on the weight of the drug substance containing amine.

Content of total impurities, in the storage-stable compositions, as determined by liquid chromatography, is not more than 3%, preferably not more than 2.5%, more preferably not more than 2%, and most preferably not more than 1.5%.

Dissolution of the drug substance containing amine, from the storage-stable compositions was studied a dissolution media selected from 0.01N hydrochloric acid (0.01N HCl), 0.1N hydrochloric acid (0.1N HCl), acetate buffer pH 4.5, phosphate buffer pH 6.8, and water. For storage-stable compositions comprising varenicline, the dissolution study was conducted in 500 ml of the dissolution media using the USP-Type I (Basket) apparatus, at 100 rpm.

In an embodiment, the dissolution of varenicline, from the storage-stable compositions, in 30 minutes, is from about 85% to about 110%, preferably from about 90% to about 110%, more preferably from about 95% to about 110%, and most preferably from about 98% to about 110%.

Storage stability of the storage-stable compositions of the present invention was studied in one or more of the following conditions:
i) 40° C.±2° C. and 75%±5% relative humidity (40° C./75% RH)
ii) 25° C.±2° C. and 60%±5% relative humidity (25° C./60% RH)
iii) 30° C.±2° C. and 75%±5% relative humidity (30° C./75% RH)

After specific time-periods of storage, the compositions were analyzed for % content of drug substance containing amine, nitrosamine content (ppm), % total impurities and average cumulative % dissolution of drug substance containing amine.

The invention is now illustrated with non-limiting examples.

### Example 1 (Comparative): Film-Coated Tablets of Varenicline Tartrate

Varenicline tartrate (0.855% w/w) and anhydrous dibasic calcium phosphate (33.5% w/w) were co-sifted through 40 mesh ASTM sieve to give a first blend. Microcrystalline cellulose (60.645% w/w), croscarmellose sodium (3% w/w) and colloidal silicon dioxide (1% w/w) were sifted through 40 mesh ASTM sieve, and geometrically mixed with the first blend to give a second blend. The second blend was mixed for 5 minutes and sifted through 40 mesh ASTM sieve and blended again for about 20 minutes. Magnesium stearate (1% w/w) was sifted through 60 mesh ASTM sieve, and mixed with the second blend for about 5 minutes, to give the lubricated powder blend.
[a] The lubricated powder blend was directly compressed into core tablets using capsular shaped, 8 mm by 4 mm, concave punch tooling, such that each tablet had a dose of 0.5 mg of varenicline.
[b] The lubricated powder blend was directly compressed into core tablets using capsular shaped, 10 mm by 5 mm, concave punch tooling, such that each tablet had a dose of 1 mg of varenicline.

Core tablets of [a] and [b] were film-coated using Opadry® (containing hypromellose 6 mPas, titanium dioxide, and polyethylene glycol) to a weight gain of 5% based on the weight of the core tablet. The coating process used only water as the solvent.

Film-coated tablets of [a] and [b] were further seal-coated using Opadry® Clear (containing hypromellose 15 mPas and triacetin), to a weight gain of 3% based on the weight of the core tablet. The coating process used only water as the solvent.

The core and seal-coated tablets of Example 1 [a] and Example 1 [b] were evaluated for content of nitrites, nitrates, and nitrosamine impurity (N-nitroso-varenicline (NNV)), the results of which are provided in Table 1.

TABLE 1
Evaluation of the Film-coated tablets of Example 1

| | |
|---|---|
| | Nitrosamine Impurity |
| Composition | (NNV) Content (ppm) |

Comparative Example 1[a]

| | |
|---|---|
| Core Tablets | 46.9 |
| Seal-Coated Tablets | 319.0 |

Comparative Example 1[b]

| | |
|---|---|
| Core Tablets | 22.5 |
| Seal-Coated Tablets | 211.7 |

As seen in Table 1, tablets of Comparative Example 1, prepared using a coating process that used only water as the solvent, showed a nitrosamine impurity (NNV) level much higher than the “acceptable level” of 18.5 ppm.

### Example 2: Film-Coated Tablets of Varenicline Tartrate (0.5 mg Varenicline)

Varenicline tartrate (0.855% w/w) and anhydrous dibasic calcium phosphate (nitrite content of 0.21 ppm, nitrate content of 3.37 ppm) (33.5% w/w) were co-sifted through 40 mesh ASTM sieve to give a first blend. Microcrystalline cellulose (nitrite content of 0.31 ppm, nitrate content of 1.88 ppm) (60.645% w/w), croscarmellose sodium (nitrite content of 0.24 ppm; nitrate content of 1.34 ppm) (3% w/w) and colloidal silicon dioxide (1% w/w) were sifted through 40 mesh ASTM sieve, and geometrically mixed with the first blend to give a second blend. The second blend was mixed for 5 minutes and sifted through 40 mesh ASTM sieve and blended again for about 20 minutes. Magnesium stearate (1% w/w) was sifted through 60 mesh ASTM sieve, and mixed with the second blend for about 5 minutes, to give a lubricated powder blend. The lubricated powder blend was directly compressed into core tablets using capsular shaped, 8 mm by 4 mm, concave punch tooling, such that each tablet had a dose of 0.5 mg of varenicline.

Core tablets were film-coated using Opadry® (containing hypromellose 6 mPas, hydroxypropyl cellulose, talc, and titanium dioxide) (nitrite content of 0.32 ppm) (to a weight gain of 8% based on the weight of the core tablet. The coating process used a solvent system consisting of isopropanol (non-aqueous solvent) (nitrite content of 0.72 ppm and nitrate content of 0.45 ppm) and water (nitrite content of 0.23 ppm and nitrate content of 0.12 ppm), in the ratio of 85:15 (water is 15% by weight of the solvent system).

Assay of varenicline, in the film coated tablets of Example 2, was within the acceptable criteria of 90% to 110%, and % cumulative dissolution in 0.01N HCl at 15 minutes was found to be about 93%. Content of nitrite, nitrate, NNV (nitrosamine impurity), and total impurities of film-coated tablets of Example 2 is provided in Table 2.

TABLE 2
Evaluation of the Film-coated tablets of Example 2

| Nitrite | Nitrate | Nitrosamine | Total |
|---|---|---|---|
| Content | Content | Impurity (NNV) | Impurities |
| (ppm) | (ppm) | Content (ppm) | Content (%) |
| 0.66 | 4.08 | 5.91 | ≤0.05 |

Tablet compositions prepared in accordance with Example 2 were packed in the following packs and subjected to storage stability studies:
i) HDPE Bottles (with 1 g molecular sieve)
ii) PVC-PCTFE (polychlorotrifluoroethylene) Blister (free of Nitrocellulose)

After different periods of storage, the tablets were analyzed for NNV (nitrosamine impurity) content, the results of which are given in Table 3.

TABLE 3
Storage Study evaluation of the Film-coated tablets of Example 2

| | | | Nitrosamine |
|---|---|---|---|
| | | Condition/ | Impurity (NNV) |
| | Pack | Time-Point | Content (ppm) |
| Initial | | | 5.91 |
| | HDPE Bottle | 25° C./60% RH/1M | 12.29 |
| | pack with 1 g | 40° C./75% RH/3M | 16.50 |
| | molecular sieve | 40° C./75% RH/6M | 15.18 |
| | PVC-PCTFE | 25° C./60% RH/7M | 10.14 |
| | Blister pack | 40° C./75% RH/3M | 9.10 |
| | | 40° C./75% RH/7M | 10.64 |

### Example 3: Film-Coated Tablets of Varenicline Tartrate (0.5 mg Varenicline)

Core tablets were prepared in accordance with Example 2. Core tablets were film-coated using Opadry® (containing hypromellose 6 mPas, hydroxypropyl cellulose, talc, and titanium dioxide) (nitrite content of 0.32 ppm) to a weight gain of 8% based on the weight of the core tablet. The coating process used a mixture of isopropyl alcohol (non-aqueous solvent) and methylene dichloride (non-aqueous solvent), in the ratio of 1:1. Tablets of Example 3 were evaluated, the results of which are provided in Table 4.

TABLE 4
Evaluation of the Film-coated tablets of Example 3

| | | Nitrosamine | | | Cumulative |
|---|---|---|---|---|---|
| | | Impurity | | | Dissolution |
| Nitrite | Nitrate | (NNV) | | Total | in 0.01N |
| Content | Content | Content | Assay | Impurities | HCl at 15 |
| (ppm) | (ppm) | (ppm) | (%) | (%) | minutes (%) |
| 1.09 | 2.26 | 4.19 | 93.9 | ≤0.05 | 92 |

Film-coated tablets of Example 3 were packed in HDPE Bottles with 1 g molecular sieve, and PVC-PCTFE (nitrocellulose free) blister packs, and analyzed for nitrosamine impurity (NNV) content after storage at 40° C./75% RH for 3 months. The results are provided in Table 5.

TABLE 5
Nitrosamine impurity content in Tablets of Example 3 on storage

| | NNV (nitrosamine impurity) Content (ppm) | |
|---|---|---|
| Condition/ | HDPE Bottle pack with | PVC-PCTFE |
| Time-Point | 1 g molecular sieve | Blister pack |
| Initial | 4.19 | |
| 40° C./75% RH/3M | 5.87 | 5.63 |

### Example 4: Film-Coated Tablets of Varenicline Tartrate (0.5 mg Varenicline)

Varenicline tartrate (0.855% w/w) and microcrystalline cellulose (nitrite content of 0.31 ppm; nitrate content of 1.88 ppm) (64.645% w/w) were co-sifted through 40 mesh ASTM sieve to give a first blend. Maltodextrin (nitrite content of 0.56 ppm; nitrate content of 3.15 ppm) (30.5% w/w) and croscarmellose sodium (nitrite content of 0.24 ppm, nitrate content of 1.34 ppm) (3% w/w) were sifted through 40 mesh ASTM sieve, and geometrically mixed with the first blend to give a second blend. The second blend was mixed for 5 minutes and sifted through 40 mesh ASTM sieve and blended again for about 20 minutes. Stearic acid (1% w/w) was sifted through a 60 mesh ASTM sieve, and mixed with the second blend for about 5 minutes, to give the lubricated powder blend. The lubricated powder blend was directly compressed into core tablets using capsular shaped, 8 mm by 4 mm, concave punch tooling, such that each tablet had a dose of 0.5 mg of varenicline.

Core tablets were film-coated using Opadry® (containing hypromellose 6 mPas, hydroxypropyl cellulose, talc, and titanium dioxide) (nitrite content of 0.32 ppm) to a weight gain of 8% based on the weight of the core tablet. The coating process used a mixture isopropyl alcohol (non-aqueous solvent) (nitrite content of 0.72 ppm and nitrate content of 0.45 ppm) and water (nitrite content of 0.12 ppm and nitrate content of 1.13 ppm), in the ratio of 85:15 (15% by weight of water).

Assay of varenicline, in the film coated tablets of Example 4, was within the acceptable criteria of 90% to 110%. Content of nitrite, nitrate, NNV (nitrosamine impurity), and total impurities of film-coated tablets of Example 4 are provided in Table 6.

TABLE 6
Evaluation of the Film-coated tablets of Example 4

| Nitrite | Nitrate | Nitrosamine | Total |
|---|---|---|---|
| Content | Content | Impurity (NNV) | Impurities |
| (ppm) | (ppm) | Content (ppm) | (%) |
| 0.66 | 3.80 | 0.53 | ≤0.05 |

Film-coated tablets of Example 4 were packed in HDPE Bottles with 1 g silica gel canister and HDPE Bottles with 1 g molecular sieve, and analyzed for nitrosamine impurity (NNV) content after storage at 40° C./75% RH. The results are provided in Table 6.

TABLE 6
Nitrosamine impurity content in Tablets of Example 4 on storage

| | Nitrosamine Impurity | |
|---|---|---|
| | (NNV) Content (ppm) | |
| Condition/ | HDPE Bottle pack with | HDPE Bottle pack with |
| Time-Point | 1 g silica gel | 1 g molecular sieve |
| Initial | 0.53 | |
| 40° C./75% RH/5M | 3.80 | 11.80 |

### Example 5: Film-Coated Tablets of Varenicline Tartrate (1 mg Varenicline and 0.5 mg Varenicline)

Lubricated powder blend, containing microcrystalline cellulose (nitrite content of 0.59 ppm and nitrate 1.80 ppm), anhydrous dibasic calcium phosphate (nitrite content of 0.22 ppm and nitrate content of 2.84 ppm), croscarmellose sodium (nitrite content of 0.15 ppm and nitrate content of 0.36 ppm), colloidal silicon dioxide (nitrite content of 1.49 ppm and nitrate content of 2.87 ppm), and magnesium stearate (nitrite content of 0.70 ppm and nitrate content of 4.45 ppm) was prepared in accordance with Example 2. The lubricated powder blend was directly compressed into core tablets having a dose of 0.5 mg using capsular shaped, 8 mm by 4 mm concave punch tooling, and into core tablets having a dose of 1 mg using capsular shaped 10 mm by 5 mm concave punch tooling.

Core tablets were film-coated using Opadry® to a weight gain of 8% based on the weight of the core tablet. The coating process used a solvent system consisting of isopropanol (non-aqueous solvent) (nitrite content of 0.36 ppm and nitrate content of 0.86 ppm) and water, in the ratio of 85:15 (water is 15% by weight of the solvent system).

Film-coated tablets of Example 5 were packed in 40 cc HDPE bottles with 1 g molecular sieve and in PVC-PCTFE Blister pack blisters. The products in the packs were analyzed for nitrosamine impurity (NNV) content after storage at 40° C./75% RH. The results are provided in Table 7.

TABLE 7
Nitrosamine impurity content in Tablets of Example 5 on storage

| | | | | |
|---|---|---|---|---|
| | Nitrosamine | | Cumulative | |
| | Impurity | | Dissolution | |
| | (NNV) | | in 0.01N | Total |
| Condition/ | Content | Assay | HCl at 15 | Impurities |
| Time-Point | (ppm) | (%) | minutes (%) | (%) |

Fim-coated Varenicline Tablets 0.5 mg packed
in HDPE Bottle pack with 1 g molecular sieve

| | | | | |
|---|---|---|---|---|
| Initial | 2.14 | 100.2 | 102 | 0.05 |
| 25° C./60% RH/6M | 1.47 | 96.9 | 102 | 0.08 |
| 30° C./75% RH/6M | 1.73 | 96.3 | 102 | 0.08 |
| 40° C./75% RH/6M | 2.18 | 99.8 | 98 | 0.15 |

Fim-coated Varenicline Tablets 0.5 mg packed
in PVC-PCTFE Blisters

| | | | | |
|---|---|---|---|---|
| Initial | 2.34 | 100.2 | 102 | 0.05 |
| 25° C./60% RH/6M | 1.82 | 100.0 | 100 | 0.15 |
| 30° C./75% RH/6M | 2.75 | 99.1 | 98 | 0.35 |
| 40° C./75% RH/6M | 4.77 | 96.4 | 95 | 1.06 |

Fim-coated Tablets 1 mg Varenicline packed
in HDPE Bottle pack with 1 g molecular sieve

| | | | | |
|---|---|---|---|---|
| Initial | 2.54 | 98.5 | 100 | ≤0.05 |
| 25° C./60% RH/6M | 1.29 | 97.9 | 99 | ≤0.05 |
| 30° C./75% RH/6M | 2.64 | 98.8 | 102 | ≤0.05 |
| 40° C./75% RH/6M | 7.6 | 98.9 | 100 | 0.08 |

Fim-coated Tablets 1 mg Varenicline packed
in PVC-PCTFE Blisters

| | | | | |
|---|---|---|---|---|
| Initial | 2.70 | 98.5 | 100 | ≤0.05 |
| 25° C./60% RH/6M | 2.64 | 98.7 | 100 | ≤0.05 |
| 30° C./75% RH/6M | 3.86 | 97.5 | 100 | ≤0.05 |
| 40° C./75% RH/6M | 6.67 | 97.0 | 97 | 0.08 |

Examples 2-5 show that pharmaceutical compositions of the present invention, comprising a drug substance containing amine and at least one excipient, and prepared by processes wherein the solvent system used in any of the process steps contains not more than about 20% by weight of water, are storage-stable and provide acceptable levels of nitrosamine impurities. Further, examples 2-5 show that the pharmaceutical compositions having reduced nitrite and nitrate content, are storage-stable and provide acceptable levels of nitrosamine impurities. The said compositions also provide the desired dissolution, assay, and total impurity content.

1. Storage-stable pharmaceutical compositions comprising drug substance containing amine and at least one excipient,
wherein the nitrite content of the compositions is not more than about 2 ppm, and
wherein the nitrosamine impurity content of the compositions is not more than about 18.5 ppm.

2. Storage-stable pharmaceutical compositions of claim 1,
wherein the compositions are manufactured by a process that uses a solvent system containing about 0% to about 20% by weight of water.

3. (canceled)

4. The storage-stable compositions as claimed in claim 1, wherein the nitrate content of the compositions is not more than about 5 ppm.

5-6. (canceled)

7. The storage-stable compositions as claimed in claim 1, wherein the excipient has a nitrite content of not more than 2 ppm.

8. The storage-stable compositions as claimed in claim 1, wherein the excipient has a nitrate content of less than or equal to 5 ppm.

9-16. (canceled)

17. The storage-stable pharmaceutical compositions of claim 1 wherein the solvent system is a mixture of at least two non-aqueous solvents, in a weight ratio ranging from 20:1 to 1:20.

18-37. (canceled)

38. Storage stable pharmaceutical compositions comprising varenicline and at least one excipient,
wherein the nitrite content of the compositions is not more than about 2 ppm, and wherein the nitrosamine impurity content of the compositions is not more than about 18.5 ppm,
wherein the nitrite content of each of excipients used is not more than about 2 ppm; and
wherein the compositions are manufactured by a process that uses a solvent system containing about 0% to about 20% by weight of water.

39. The storage-stable pharmaceutical compositions of claim 1, wherein the nitrate content of the said compositions is not more than about 5 ppm.

40. Storage-stable pharmaceutical compositions comprising varenicline and diluent from about 60% to about 95% by weight of the composition,
disintegrant from about 1% to about 20% by weight of the composition,
binder from about 0.1% to about 20% by weight of the composition,
lubricant from about 0.25% to about 5% by weight of the composition,
glidant from about 0.1% to about 10% by weight of the composition,
optionally coating agent,
wherein the nitrite content of the compositions is not more than about 2 ppm, and
wherein the nitrosamine impurity content of the compositions is not more than about 18.5 ppm; and
wherein the compositions are manufactured by a process that uses a solvent system containing mixture of at least one non-aqueous solvent and water, in a weight ratio ranging from 4:1 to 1:0.

41. The storage-stable pharmaceutical compositions of claim 8,
wherein the diluent is selected from microcrystalline cellulose, starch, calcium phosphate, dibasic calcium phosphate, tribasic calcium phosphate, mannitol, sorbitol, lactose, and mixtures thereof,
wherein the disintegrant is selected from sodium starch glycolate, starch, pregelatinized starch, crospovidone, croscarmellose sodium, and mixtures thereof,
wherein the binder is selected from hydroxypropyl methyl cellulose (HPMC), hydroxypropyl cellulose (HPC), methylcellulose, hydroxyethyl cellulose, starch, polyvinylpyrrolidone (PVP), polyvinyl alcohol, waxes, fatty alcohols, polyethylene glycol, carbomer, gums, alginates, polymethacrylates, and mixtures thereof,
wherein the lubricant is selected from magnesium stearate, calcium stearate, stearic acid, sodium stearyl fumarate, sodium benzoate, palmitic acid, talc, and glyceryl behenate, and
wherein the glidant is selected from colloidal silicon dioxide, hydrated silicon dioxide, light anhydrous silicic acid, aluminium silicate, titanium oxide, stearic acid, and talc.

42. Storage-stable pharmaceutical compositions comprising uncoated tablet comprising:
a) 0.855% w/w of varenicline;
b) 33.5% w/w of anhydrous dibasic calcium phosphate;
c) 60.645% w/w of microcrystalline cellulose;
d) 3.000% w/w of croscarmellose sodium;
e) 1.000% w/w of colloidal silicon dioxide; and
f) 1.000% w/w of magnesium stearate;
wherein uncoated tablet is coated with opadry coating,
wherein the nitrite content of the compositions is not more than about 2 ppm, and wherein the nitrosamine impurity content of the compositions is not more than about 18.5 ppm, and
wherein the nitrite content of each of excipients used is not more than about 2 ppm; and
wherein the compositions are manufactured by a process that uses a solvent system containing about 0% to about 20% by weight of water.
